(19) United States
(12) Patent Application Publication
(10) Pub. No.: US 2020/0352835 A1
(43) Pub. Date: Nov. 12, 2020

(54) COMPOSITIONS FOR PROTECTING AND REPAIRING SKIN

(71) Applicant: KEMIN INDUSTRIES, INC., Des Moines, IA (US)

(72) Inventors: Kelli Herrlinger, Ankeny, IA (US); Joanne Lasrado, Des Moines, IA (US); Paul Falcone, Des Moines, IA (US); Richard Roberts, Johnston, IA (US); Filipa Quintela, Des Moines, IA (US); Samanta Maci, Belas (PT)

(21) Appl. No.: 16/868,517

(22) Filed: May 6, 2020

Related U.S. Application Data
(60) Provisional application No. 62/844,116, filed on May 6, 2019.

(51) Int. Cl. A61K 8/34; A61Q 19/02; A61Q 17/04

(52) U.S. Cl. CPC A61K 8/345; A61Q 17/04; A61Q 19/02

(57) ABSTRACT

The present invention relates generally to methods for protecting human skin against light damage and, more specifically, to the use lutein and/or zeaxanthin to protect human skin from damage by exposure to blue light and white light. Another aspect of the present invention relates to methods for repairing damaged skin cells, including mitigating the damage caused by exposure to light or other stressors.

### RELATED APPLICATION

This application claims the benefit of priority to U.S. Patent Application Ser. No. 62/844,116, filed May 6, 2019, entitled “COMPOSITIONS FOR PROTECTING AND REPAIRING SKIN,” the entire disclosure of which is incorporated herein by reference.

### BACKGROUND OF THE INVENTION

The electromagnetic energy in the solar spectrum that reaches earth surface includes UV radiation, visible light and infrared radiation. The effect of light and radiation on skin and tissues has been studied extensively in the past especially the use of light and radiation irradiation sources for treating pathological dermatology conditions (Frain-Bell W. The effect of light on the skin. British Journal of Dermatology. 1988, 119:479-485). Light travels as photons and the absorption of photons by a molecular structure in the physiological system initiates the photobiological reactions (Rupert C. S. The biological effectiveness of Ultraviolet light. National cancer institute monograph. 1978, 50:85-89. ISSN: 0083-1921).

Visible light, or white light, comprises the region of light within the wavelengths of 400-750 nm in the electromagnetic spectrum. Blue light is the portion of the electromagnetic spectrum in the visible region with wavelengths ranging from 400-500 nm. The wavelengths of blue light are close to the shorter ultraviolet wavelengths, typically called the UVA spectrum (315-400 nm), and the blue region of the visible spectrum is particularly important because it still has high energy (even though lower than UV) and longer wavelengths that can penetrate tissue deeper than UV light. Godley B. F., Shamsi F. A., Liang F. Q., Jarrett S. G., Davies S. and Boulton M., Blue light induces mitochondrial DNA damage and free radical production in epithelial cells, J. Biol. Chem. 280 (2005) 21061-21066; Opländer C., Hidding S., Frauke B., Werners F. B., Born M., Pallua N. and Suschek C. V. Effects of blue light irradiation on human dermal fibroblasts. Journal of Photochemistry and photobiology B: Biology. 2011. 103: 118-125.

The deleterious effects of skin exposure to ultraviolet (UV) radiation (both UVA and UVB) are well known. The UV radiation is categorized into three regions, UVC, UVB and UVA. Most of the UVC is filtered by ozone layer and high energy shorter wavelength UVB get absorbed in the upper layers of skin, i.e. epidermal region, while UVA penetrates little deeper into dermal regions of the skin. To date, most studies on skin exposure to light have concentrated on the role of UV irradiation due to its high energy, photo reactivity and its associated damage to the skin while the role of visible light has been less extensively investigated (Mahmoud B. H., Hexsel C. L., Hamzavi I. H. and Lim H. W. Effects of Visible Light on the Skin. Photo chem Photobiol, 2008, 84: 450-462). The effect of visible light on skin in terms of cytokine release, matrix metalloproteinase (MMPs) production was investigated by Liebel et al and few others, but the effects of blue light on skin was not explored extensively (Liebel F, Kaur S, Ruvolo E, Kollias N and Southall M. D. Irradiation of Skin with Visible Light Induces Reactive Oxygen Species and Matrix-Degrading Enzymes. Journal of Investigative Dermatology. 2012. 132: 1901-1907; doi:10.1038/jid.2011.476). Thus, there remains a need to investigate the effect of exposure to light on skin and identify compositions capable of conferring protective properties.

### SUMMARY OF THE INVENTION

The present invention relates generally to methods for protecting human skin against light damage and, more specifically, to the use of FloraGLO® Lutein to protect human skin from damage by exposure to blue light and white light. Another aspect of the present invention relates to methods for repairing damaged skin cells, including mitigating the damage caused by exposure to light or other stressors.

### DETAILED DESCRIPTION OF THE FIGURES

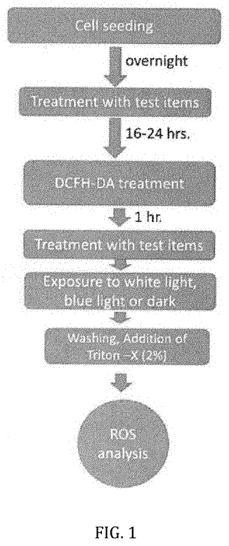

FIG. 1 depicts the protocol for evaluating the antioxidant effect of FloraGLO Lutein against blue light and white light.

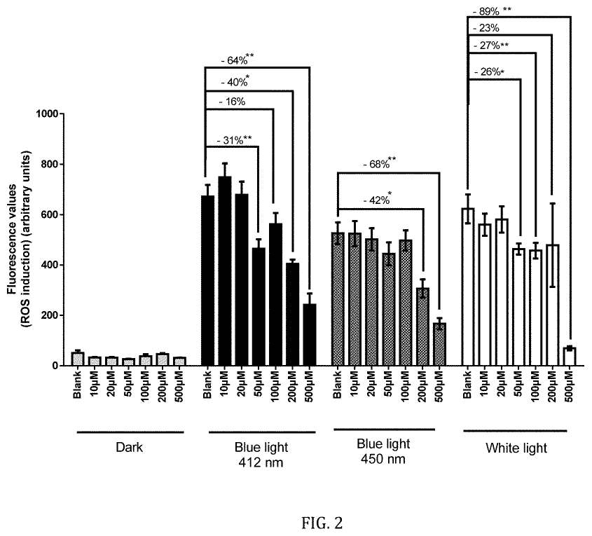

FIG. 2 depicts the reactive oxygen stress (ROS) after exposure to blue light (412 nm, 450 nm) and white light. The values represent the % inhibition compared to the respective blank.

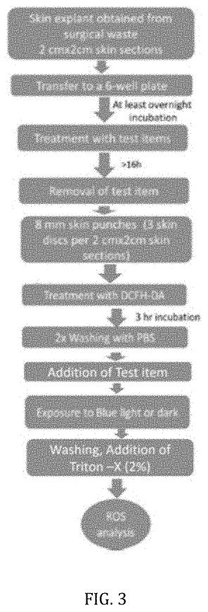

FIG. 3 depicts the protocol for evaluating skin explants against the oxidative stress induced by blue light and white light.

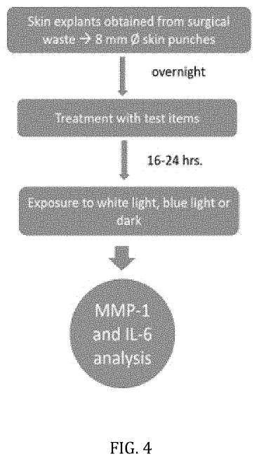

FIG. 4 depicts the protocol for evaluating skin explants against the inflammation induced by blue light and white light.

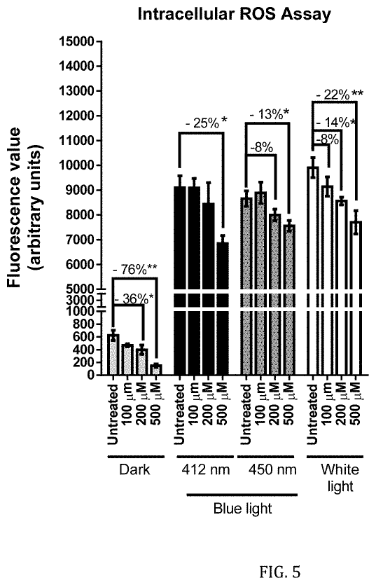

FIG. 5 depicts the effect of different concentrations of FloraGLO Lutein on the level of ROS. The fluorescence values are proportional to ROS induction. * corresponds to p<0.05 and ** for student p<0.01. The following P value was obtained after One-way ANOVA analysis of the different groups: Dark: p=0.0023; Blue light 412 nm: p=0.0620; Blue light 450 nm: p=0.0568; White light: p=0.0177.

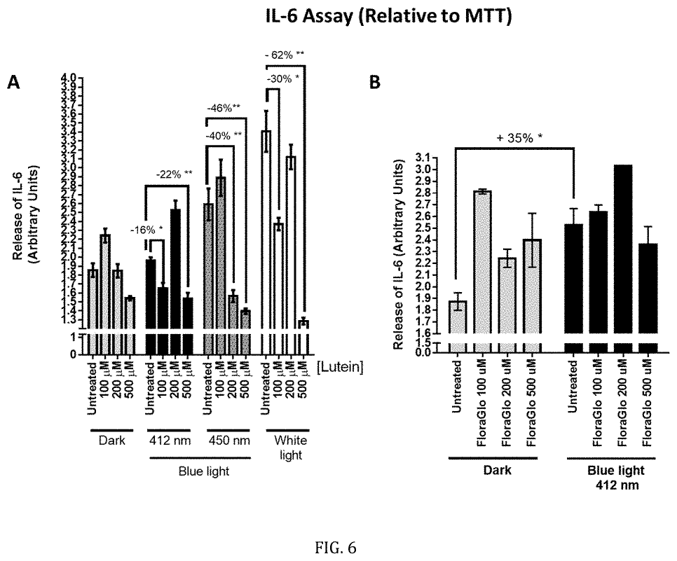

FIG. 6 depicts the effect of different concentrations of FloraGLO Lutein on IL-6 release. (A) represents Run 1 and (B) represents Run 2. The absorbance was measured at 450 nm using a Synergy HTX multimode reader. The concentrations were calculated based on the absorbance of IL-6 assay and relative to skin viability (MTT) results. * corresponds to p values <0.05 and ** for p values <0.01. The following P value was obtained after One-way ANOVA analysis of the different groups: RUN 1 (A): Dark: p=0.0006; Blue light 412 nm: p<0.0001; Blue light 450 nm: p=0.0001; White light: p<0.0001. RUN 2 (B) dark: p=0.0444; Blue light 412 nm: p=0.0135.

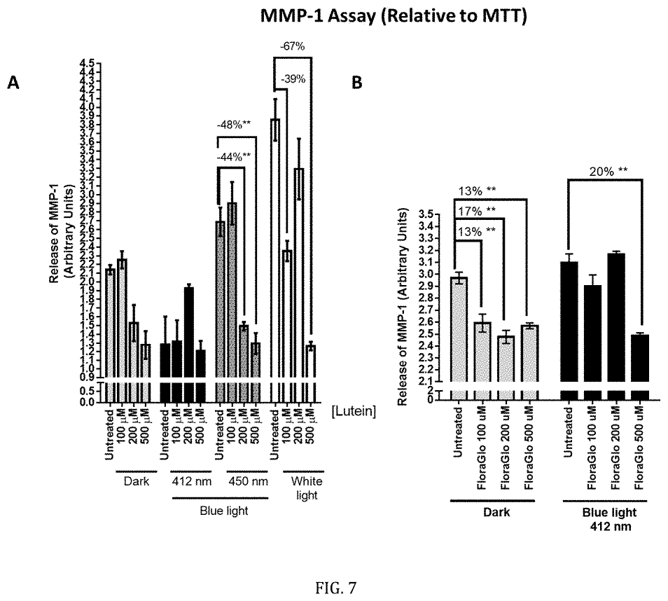

FIG. 7 depicts the effect of different concentrations of FloraGLO Lutein on MMP-1 release. (A) represents Run 1 and (B) represents Run 2. The absorbance was measured at 450 nm using a Synergy HTX multimode reader. The concentrations were calculated based on the absorbance of MMP-1 assay and relative to skin viability (MTT) results * corresponds to student p values <0.05 and ** for p values <0.01. The following P value was obtained after One-way ANOVA analysis of the different groups: RUN 1 (A) Dark: p=0.0032; Blue light 412 nm: p=0.0689; Blue light 450 nm: p=0.0002; White light: p=0.0123. RUN 2 (B) dark: p=0.0009; Blue light 412 nm: p=0.0002.

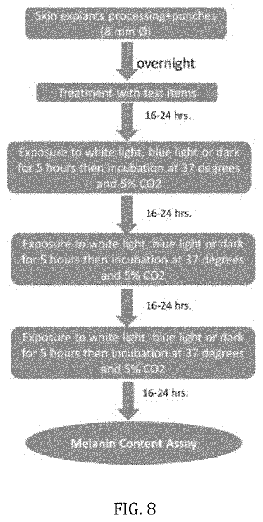

FIG. 8 depicts the protocol for evaluating against the pigmenting effect of blue light on human skin explants.

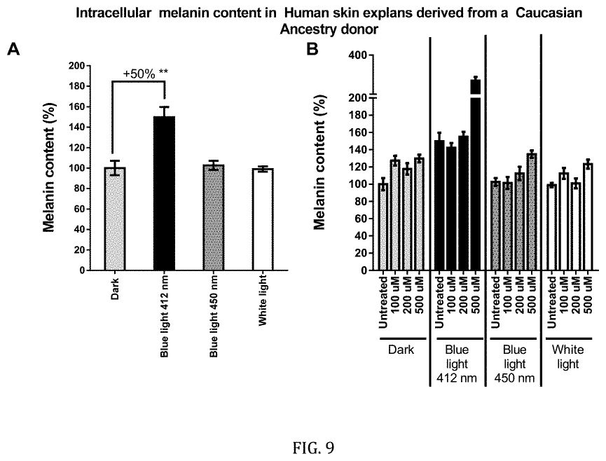

FIG. 9 describes the effect of different concentrations of FloraGLO Lutein on the pigmenting effect of blue light on human skin explants derived from a Caucasian ancestry donor. Left panel: The effect of light on the melanin content was monitored on human skin explants independent of FloraGLO Lutein treatment. * corresponds to p<0.05 and ** for p<0.01. The P value of the overall experiment was evaluated using One-way ANOVA analysis. The result is p<0.0001. Right panel: The effect of light on the melanin content was monitored on human skin explants exposed to different concentrations of FloraGLO Lutein.

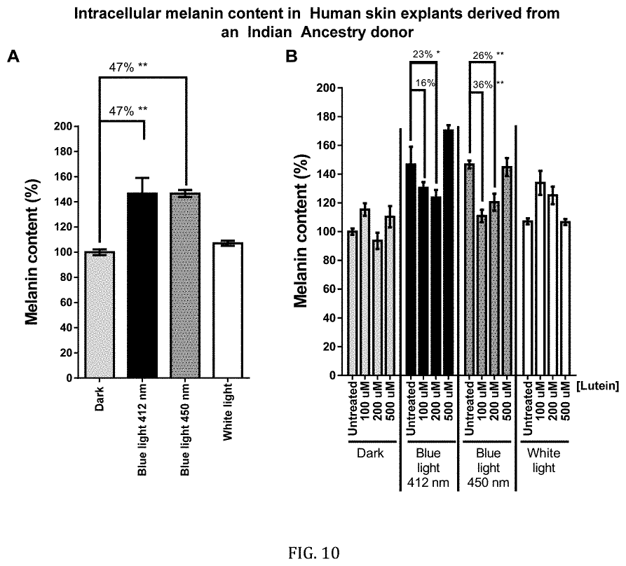

FIG. 10 describes the effect of different concentrations of FloraGLO Lutein on the pigmenting effect of blue light on human skin explants derived from an Indian ancestry donor. Left panel: The effect of light on the melanin content was monitored on human skin explants not exposed to FloraGLO Lutein. * corresponds to p<0.05 and ** for p<0.01. The P value of the overall experiment was evaluated using One-way ANOVA analysis. The result is p<0.0001. Right panel: The effect of light on the melanin content was monitored on human skin explants exposed to different concentrations of FloraGLO Lutein.

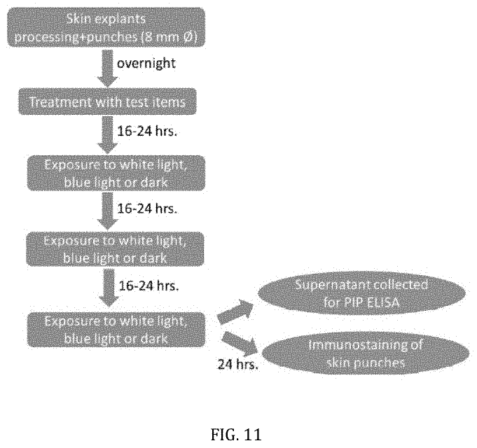

FIG. 11 depicts the protocol for evaluating against the detrimental effect of blue light on skin collagen.

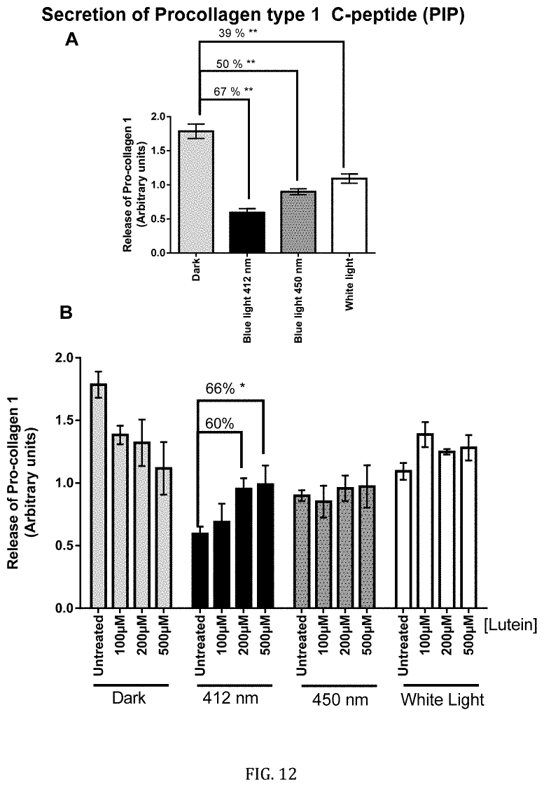

FIG. 12 describes the effect of different concentrations of FloraGLO Lutein on the secretion of Procollagen type I C. * corresponds top <0.05 and ** for p<0.01. (A) represents the level of Procollagen I after no exposure to light or after exposure to blue light or white light. The P value of the overall experiment was evaluated using One-way ANOVA analysis. The result is p<0.0001. (B) represents the level of Procollagen I in skin explants treated with FloraGLO® Lutein and exposed to light.

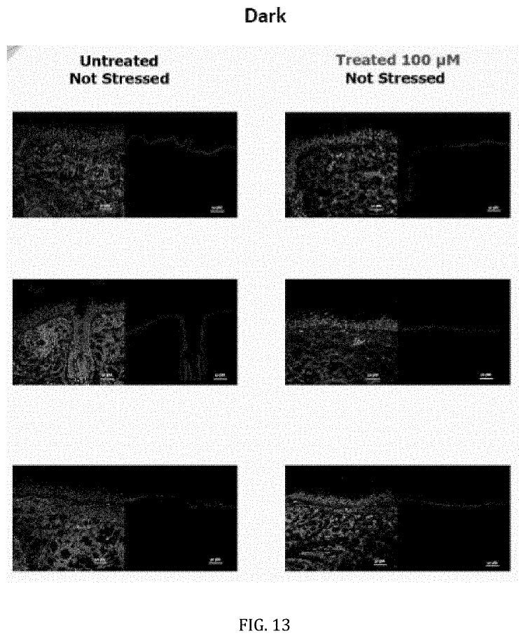

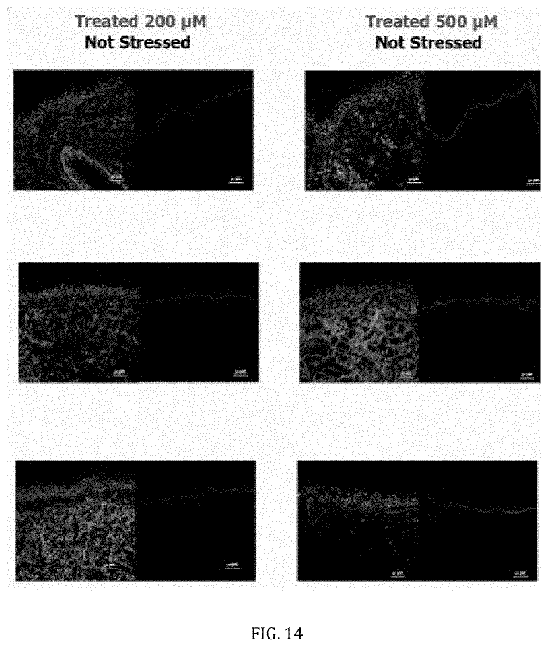

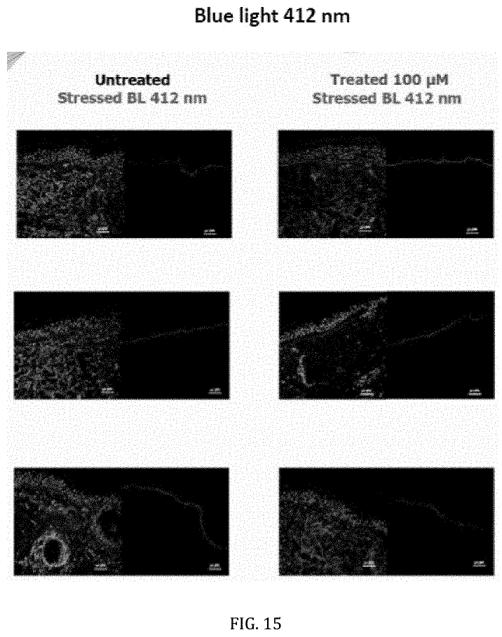

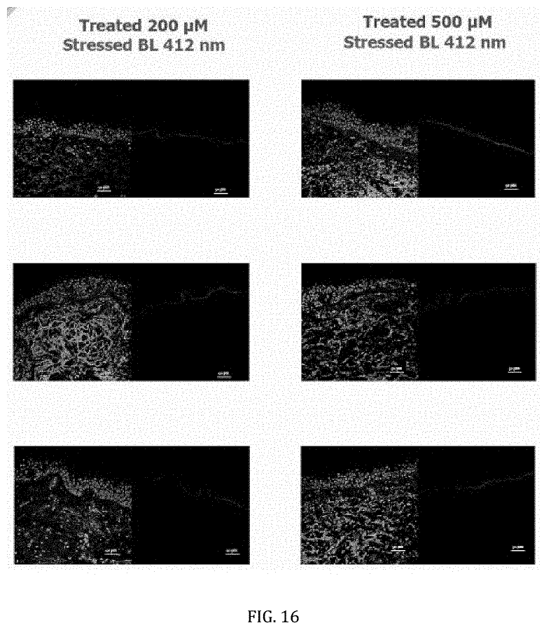

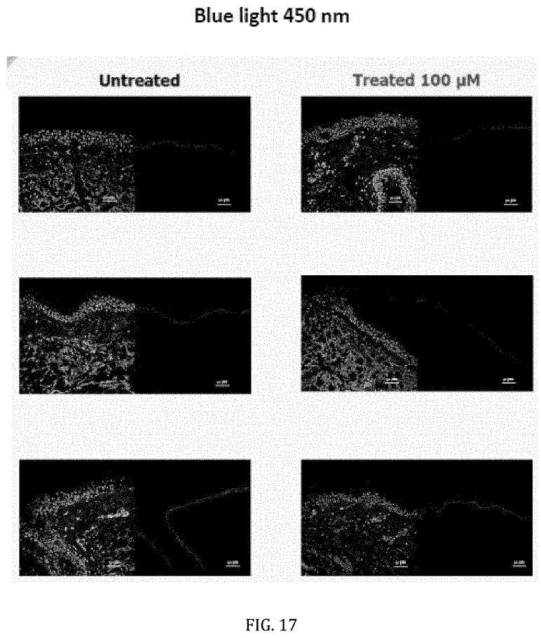

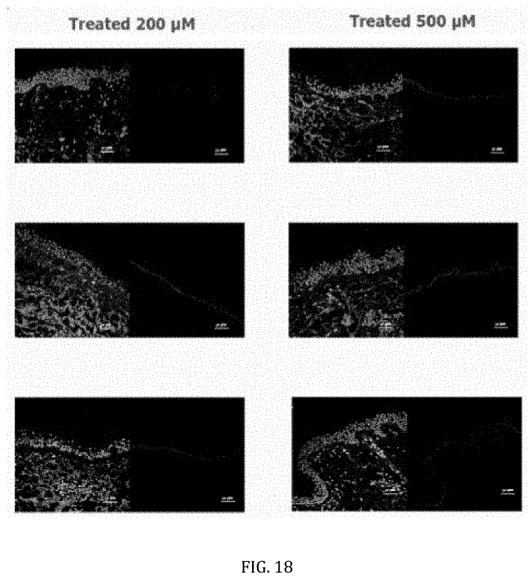

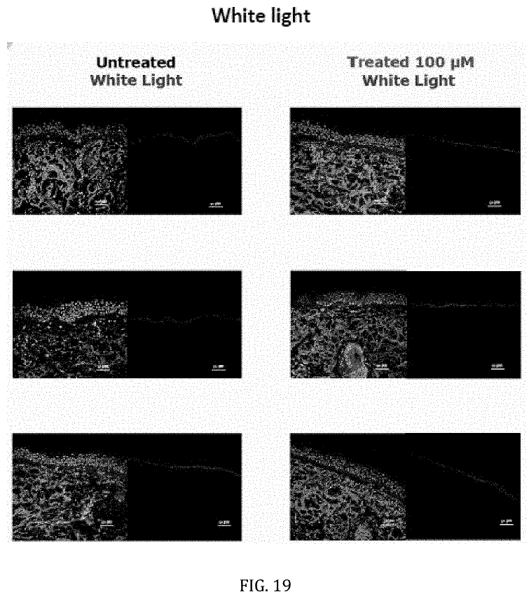

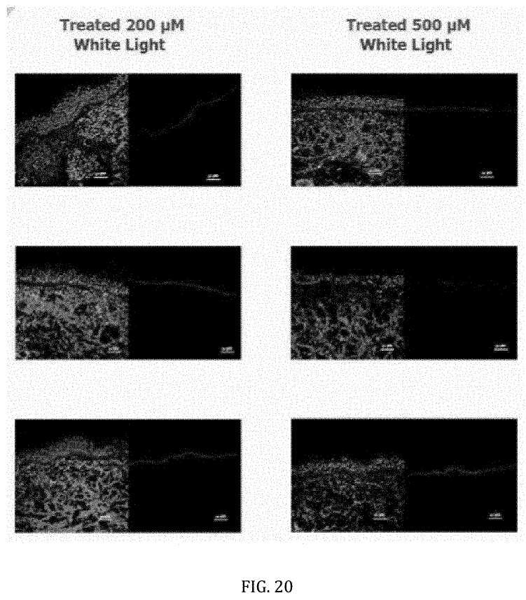

FIGS. 13-20 describe the effect of different concentrations of FloraGLO Lutein on the level of collagen at the dermoepithelial junction. The red staining represents staining of Collagen and the blue staining represents a DAPI staining (staining of nucleus). A red Sirius staining (stains collagen was performed on the skin explants and the skin integrity was indirectly determined. This staining allowed observation of the different layers of the epidermis, the dermo epithelial junction as well as the dermis. As shown in FIG. 13-20, in skin explants treated FloraGLO Lutein, no disruption in the skin integrity could be observed.

### DETAILED DESCRIPTION OF THE INVENTION

The present invention relates generally to methods for protecting human skin against light damage and, more specifically, to the use of FloraGLO Lutein to protect human skin from damage by exposure to blue light and white light. Another aspect of the present invention relates to the unexpected benefits of using these methods to repair or mitigate damaged skin cells, including damage caused by exposure to light or other stressors.

Skin undergoes stress induced by exposure to visible and ultraviolet radiations (UV) from sun. In addition, advances in technology such as the prevalence of LED lights and the blue light emitted from electronic devices such as mobile phones and tablets, has dramatically increased human exposure, and potential skin damage, due to blue light.

As a body's natural response to environmental stress, skin cells release a vast array of cytokines and proteolytic enzymes such as matrix metalloproteinase (MMPs), which lead to the breakdown of collagen and other extracellular matrix proteins, ultimately leading to appearance of fine lines and wrinkles.

The inventors investigated the effect of blue light (at two wavelengths) and white light on skin cells and explored the protective effect of compositions comprising FloraGLO Lutein against damage caused by exposure to blue light and white light. The inventors also investigated whether there were any changes to the skin cells in the dark.

Human keratinocytes cells (upper skin layer) and skin explants (epidermis and dermis) were treated with FloraGLO® Lutein (Kemin Industries, Inc., Des Moines, Iowa). A range of different concentrations were used to screen and identify efficacy at different dosage levels. Across the studies, the skin explants and cells were exposed to the following four environmental conditions:
Violet/Blue light at 412 nm—high energy close to ultra-violet (UV)
Blue light at 450 nm—proximately in the middle of the blue light wavelength range
White light—represents the full visible (including blue light) light spectrum which mimics our everyday exposure to sunlight
Dark

### EXAMPLE 1

General materials and analytical methods. This experiment was designed to evaluate the ability of FloraGLO® Lutein to quench Reactive Oxygen Species (ROS) after exposure to white light and blue light at two wavelengths (412 nm and 450 nm). The protocol is described in FIG. 1. In this study, several concentrations were tested (10 μM, 20 μM, 50 μM, 100 μM, 200 μM, and 500 μM). The oxidative stress was measured through DCFH-DA method.

After incubation, the cells were washed with PBS and were then treated with dye 2′,7′-Dichlorofluorescein diacetate (DCFH-DA) for 1 hour at 37° C. and 5% CO2. After 1 hour of incubation, the cells were washed twice with PBS. 100 μL of the different concentrations of FloraGLO® Lutein (Kemin Industries) were added per well (except for blank) and the cells were exposed to the four environmental conditions. After the exposure period of 1 hour, the cells were washed with 500 μL of the lysis solution (2% Triton X-100) were added to all wells. Then the wellplate was shaken for 30 minutes and the fluorescence (wavelength of excitation of 485-488 nm and emission 528-530 nm) was measured using the Synergy HTX multimode microplate reader.

Results. The results indicate that the FloraGLO Lutein at concentrations of 50 μM or greater was effective in protecting the cells against ROS induced by blue light and white light, as shown in FIG. 2.

### EXAMPLE 2

General materials and analytical methods. This experiment was designed to evaluate the efficacy of FloraGLO Lutein 10% on full skin explant to inhibit the detrimental effect of blue light. The oxidative stress (monitoring of level of Reactive Oxygen Species (ROS)), aging (monitoring level of matrix metalloproteinase-1 (MMP-1)) and inflammation (monitoring level of interleukin-6 (IL-6)) were examined through different assays.

The skin explants were treated with the different concentrations of the test item and exposed to different environmental conditions (white light, Blue Light (412 nm), Blue Light (450 nm), and Dark). Each assay is described in greater detail below:

### Intracellular Reactive Oxygen Species (ROS) Scavenging Activity Assay

As described in FIG. 3, full skin explants were treated with test item and exposed to different environmental conditions. After exposure, a non-fluorescent probe, DCFH-DA was added. The DCFH-DA was taken up by cells by passive diffusion and was deacetylated by cellular esterases to non-fluorescent DCFH, which is trapped within cells. The fluorescent DCF was generated upon enzymatic reduction and subsequent oxidation by ROS. Fluorescence was measured over time and correlates to the ability to scavenge or exacerbate ROS.

The oxidative stress assay was conducted as follows:

After incubation with test items, the full skin sections were washed with PBS and 8 mm skin discs punches were made (3 discs per skin section). The discs were then treated with the dye DCFH-DA for 3 hours at 37° C. and 5% CO2. After 3 hours of incubation, the full skin discs were washed twice with PBS. 100 μL of FloraGLO® Lutein at the concentration of 100 μM, 200 μM and 500 μM were re-applied to the skin discs and t then exposed for 45-60 minutes to: White light (20 J/cm2), Blue Light 412 nm (20 J/cm2), Blue Light 450 nm (20 J/cm2) or dark. After the exposure period, the skin was washed and 500 μL of lysis solution (2% Triton X-100) were added to all wells.

The wellplate was shaken for 30 minutes and the fluorescence (maximum excitation and emission spectra of 485-488 nm and 528-530 nm respectively) was measured using the Synergy HTX multimode microplate reader.

### Collagenase Inhibition Assay (MMP-1)

As described in FIG. 4, full skin explants were treated with test item and exposed to different environmental conditions to stimulate collagenase synthesis. Twenty-four hours after exposure the supernatant was collected and transferred in an antibody coated-well (ELISA wellplate). The target protein in the supernatant binds to the immobilized antibody. A biotinylated detection antibody specific to the target protein was added to the well. After the addition of 3,3′,5,5′ Tetramethylbenzidine(TMB) substrate, a color developed proportional to the amount of target protein bound. The Stop solution changed the color from blue to yellow. The intensity of the color was then measured by spectrometry at 450 nm and its reduction is directly proportional to the test item's ability to inhibit collagenase.

### Pro-Inflammatory Cytokine Inhibition Assay

As described in FIG. 4, full skin explants were treated with test item and exposed to different environmental conditions to stimulate production and secretion of pro-inflammatory cytokines, including IL-6. Twenty-four hours after exposure the supernatant was collected and transferred in an antibody coated-well (ELISA wellplate). The target protein in the supernatant binds to the immobilized antibody. A biotinylated detection antibody specific to the target protein was added to the well. After the addition of TMB (3,3′,5,5′-Tetramethybenzidine) substrate (reagent detecting Horseradish Peroxidase), a color developed proportional to the amount of target protein bound. The Stop solution changed the color from blue to yellow. The intensity of the color was measured by spectrometry at 450 nm and is directly proportional to the concentration of IL-6 present.

Results. The results for each assay are depicted in FIGS. 5-7, showing that FloraGLO®, Lutein 10% is capable of decreasing the level of ROS induced by either blue light (both wavelength) or white light. FloraGLO Lutein is also capable of decreasing the level of the pro-inflammatory cytokines IL-6 and the collagenase MMP-1. Based on these results, FloraGLO Lutein is capable of reducing the level of ROS and exhibits anti-inflammatatory and anti-aging potential. Further still, the differences in ROS shown in the dark indicate the potential to repair skin or mitigate against damage associated with chronological aging.

### EXAMPLE 3

General materials and analytical methods. This experiment was designed to evaluate the efficacy of FloraGLO Lutein against the pigmenting effect of blue light on human skin explant, as described in FIG. 8. The melanin content of the skin explants was examined through the melanin content assay. In these experiments skin explants from 2 donors (from Caucasian and Indian ancestry) were used. Run 1 used skin explants from the Caucasian donor. Run 2 used skin explants from the donor of Indian ancestry. The same environmental conditions were used, white light, blue light at two wavelengths (412 nm, 450 nm) and dark.

Results. The intracellular melanin content in human skin explants were evaluated from Run 1 (Caucasian ancestry) and Run 2 (Indian ancestry) with the results shown in FIGS. 9 and 10. The current study did not identify changes in melanin content in the skin donated from an individual of Caucasian ancestry. However, in skin explants derived from an individual of Indian ancestry treated with media containing 100 uM and 200 uM there was a decrease in the melanin content which was induced by both blue light 412 nm and 450 nm.

### EXAMPLE 4

General materials and analytical methods. This experiment was designed to evaluate the efficacy of FloraGLO Lutein 10% on full skin explant to inhibit the detrimental effect of blue light on collagen, as described in FIG. 11. The level of procollagen type I (a precursor of type I collagen) and collagen will be examined through ELISA and immunohistochemistry respectively.

Results. The skin explants were treated with the different concentrations of the test item and exposed to different environmental conditions: white light, blue light at two wavelengths (412 nm and 450 nm), and dark.

### Procollagen Type I C-Peptide (PIP) EIA Kit

As depicted in FIG. 11, the full skin explant was treated with test item and exposed repeatedly to different environmental conditions. The supernatant was collected and transferred in an antibody coated-well (ELISA wellplate). The target protein in the supernatant binds to the immobilized antibody. A biotinylated detection antibody specific to the target protein was added to the well. After the addition of TMB substrate (substrate of the horseradish peroxidase), a color develops proportional to the amount of target protein bound. The Stop solution changes the color from blue to yellow. The intensity of the color was measured by spectrometry at 450 nm and its reduction is directly proportional to the test item's ability to inhibit collagenase.

### Monitoring Level of Collagen-Immunohistochemistry

As depicted in FIG. 11, the full skin explant was treated with test item and exposed repeatedly to different environmental conditions. The level of collagen was then monitored by immunohistochemistry.

Results. In the skin explants exposed to blue light 412 nm there was a decrease in secreted procollagen (FIG. 11). Treatment of skin explants with FloraGLO® Lutein protected against this decrease, with a significant difference observed at FloraGLO Lutein concentration of 500 μM. The researchers also observed the effect of different concentrations of FloraGLO Lutein on the level of collagen at the dermoepithelial junction as displayed in FIGS. 12-20. The researchers concluded that FloraGLO Lutein is capable of supporting and maintaining the collagen framework.

According to at least one embodiment, lutein and/or zeaxanthin is administered in an amount effective to provide protection to skin caused by oxidative damage induced by exposure to blue light or visible light (400-750 nm) conditions, for instance in an amount of at least 0.25 mg lutein. In alternative embodiments, the lutein and/or zeaxanthin is delivered as an oral administration in an amount ranging from about 0.25 to about 40 mg. In alternative embodiments, the lutein and/or zeaxanthin is administered topically in an amount of at least 10 ppm. For instance, the lutein and/or zeaxanthin is delivered as a topical application, such as a lotion or cream. The lutein could also be an oil or suspension.

According to at least one embodiment, the present invention includes a composition comprising lutein and/or zeaxanthin that is administered in an amount effective to repair damaged skin or to reduce damage caused by oxidative stress, photoaging or inflammation induced by exposure to blue light or visible light (400-750 nm) conditions.

In another embodiment, the present invention includes methods of administering lutein and/or zeaxanthin, orally or topically, in an amount effective to protect or repair the skin from damage caused by oxidative damage induced by exposure to blue light or visible light (400-750 nm) conditions. Alternative embodiments include administering lutein and/or zeaxanthin in an amount effective to repair skin or reduce the effects of chronological aging on human skin. As used herein, the term chronological aging refers to the natural aging process.

Alternative embodiments include administering lutein and/or zeaxanthin in an amount effective to repair skin or reduce the effects of photoaging. Persons of ordinary skill in the art would understand that the term photoaging falls outside the generally understood definition of chronological aging. As used herein, the term photoaging generally refers to pre-mature aging, which could be caused by exposure to light or other stressors. By way of non-limiting example, the present invention relates to compositions and methods to protect or repair the skin, or alternatively reduce the effects of photoaging induced by exposure to blue light, visible light (white light 400-750 nm), or dark conditions.

Alternative embodiments include compositions and methods of administering lutein and/or zeaxanthin, orally or topically, in an amount effective to repair the skin or in an amount effective at attenuating damage to the skin induced by blue light, visible light (white light 400-750 nm), or dark conditions. For instance, in certain embodiments, the present invention relates to administering lutein and/or zeaxanthin, orally or topically, in an amount effective to attenuate damage to the collagen framework induced by blue light exposure. The present invention also relates to compositions for attenuating skin pigmentation comprising lutein and/or zeaxanthin, for oral or topical administration, wherein the composition contains lutein in an amount capable of attenuating skin pigmentation induced by blue light exposure in dark skinned individuals. In at least one embodiment, attenuating skin pigmentation includes maintenance of an even skin tone.

In certain embodiments, the present invention is a topical application, such as a cream or lotion. For instance, a cream that can be applied overnight. In other embodiments, the lutein is delivered via a dermal patch. In other embodiments, the present invention is administered orally, for instance in a capsule, tablet, powder or suspension. Persons of ordinary skill in the art will readily appreciate the various alternative methods for administering an effective amount of lutein and/or zeaxanthin.

According to at least one embodiment, lutein and/or zeaxanthin is administered orally in a dosage amount of 0.25 mg or more. In certain embodiments, the lutein and/or zeaxanthin is administered in an amount ranging from about 0.25 to about 40 mg, for instance in an amount ranging from about 0.5 to about 25 mg.

According to a further embodiment, lutein and/or zeaxanthin is administered topically in a dosage amount of at least 10 ppm. In certain embodiments, the lutein and/or zeaxanthin is administered in an amount ranging from about 10 to 500 ppm, for instance from about 0.001 to about 0.05%, or alternatively from about 1 mg to about 50 mg in a 100 g tube of cream.

It should be further appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplishes at least all of the intended objectives.

1. A method of delivering an effective amount of lutein and/or zeaxanthin to provide protection to skin caused by damage induced by exposure to blue light, visible light (white light 400-750 nm) or dark conditions.

2. The method of claim 1, wherein the composition is an oral supplement.

3. The method of claim 2, wherein the oral supplement contains lutein in an amount that delivers at least 0.25 mg.

4. The method of claim 2, wherein the oral supplement contains lutein in an amount ranging from about 0.25 to 40 mg.

5. The method of claim 1, wherein the composition is a topical application.

6. The method of claim 5, wherein the composition is capable of delivering lutein to the skin in an amount of at least 10 ppm.

7. The method of claim 5, wherein the composition can deliver lutein to the skin in an amount that ranges about 10 to 500 ppm.

8. A composition comprising lutein and/or zeaxanthin in an amount effective to protect or repair human skin from oxidative stress, photoaging or inflammation induced by exposure to blue light, visible light (white light 400-750 nm) or dark conditions.

9. A method of mitigating or repairing damage to human skin that has been exposed to light or other stressors wherein the method comprises orally or topically administering an effective amount of lutein and/or zeaxanthin.

10. The method of claim 9, wherein the composition is applied to the skin in the form or a cream or lotion and delivered in an amount of at least 1 mg lutein.

11. The method of claim 9, wherein the composition is orally administered in an amount ranging from about 0.25 to about 40 mg.

12. A method of repairing damaged human skin or reducing the effects of chronological aging on human skin that has been exposed to light or other stressors, comprising topically administering to the damaged skin a composition that contains lutein and/or zeaxanthin in an effective amount.

13. The method of claim 12, wherein the composition is applied to the skin in the form or a cream or lotion and delivered in an amount ranging from about 10 to about 500 ppm.

14. The method of claim 12, wherein the composition is orally administered in an amount ranging from about 0.25 to about 40 mg.

15. An anti-aging composition comprising lutein and/or zeaxanthin, for oral or topical administration, wherein the lutein is administered in an amount effective to protect or mitigate damage to the skin induced by blue light, visible light (white light 400-750 nm) or dark conditions.

16. The composition of claim 15, wherein the oral or topical administration attenuates damage to the collagen framework induced by blue light exposure.

17. The method of claim 15, wherein the composition is applied to the skin in the form or a cream or lotion and delivered in an amount of at least 10 ppm.

18. The method of claim 15, wherein the composition is orally administered in an amount ranging from about 0.25 to about 40 mg.

19. A composition for attenuating skin pigmentation comprising lutein and/or zeaxanthin, for oral or topical administration, wherein the lutein is administered in an amount capable of attenuating skin pigmentation induced by blue light exposure in dark skinned individuals.

20. The composition of claim 19, wherein attenuating skin pigmentation includes maintenance of an even skin tone.
